SEQUENCE LISTING (6 sequences)

SEQ ID NO: 1 - DNA, 873 bp, Pusillimonas noertemannii
atggcttcagcggtattggcgcttgttttcgcgctgtcgggggtagcgcatggtcgggtcgtgttgccggaaccggtcaattttcctagcctggacaactggaagggcgacgcgcccttgatgctggacggctatctgttcaagccagccggacacaagccctacccggccgttgtcatgttccacggttgtgcgggcgccatatcggtcaagacggggcgcataacctcgcggttcaccaatatggcgcagctgctgaacgacatgggttatgccgtgctgatcgtcgacagcttcaatccgcgcggcgtgccgcagatctgcacggttccgttggccgaccgggacatcaagaacaggcatcgcgtgatggatgcctacagcgccttgcagtacctgaattcgcgcccggatatcgttgcgggcaagatcggcgccatagggttttcgcatggcggcagcggcgcgttgtcatcaatggatgctacaaccgaagcctatggccgttcggagcagaagtttgcggcttcggtggcactgtacccgggttgcggctggcaatcgcgcaagcagccggagttctcggcatacggccccctgttgattctggctggagaaaaggacgattggacgccggtcgagccttgtcgaaagctggcggcgcgcagccagcagcgtaacgagccggtcgagttggtcgtgtatcccgatgcctatcacgcctttgacagcaccagcgaggtctatgtgcgcaaagacgtgcccaatggtgtcaatggttcggcaggcgtgcacgtgggggccaatccggcggcccgcgaagcggcatacaaacgtatccgcgagttttttcaccagcatttgcgtccgtga

SEQ ID NO: 2 - protein, 290 aa, Pusillimonas noertemannii
MASAVLALVFALSGVAHGRVVLPEPVNFPSLDNWKGDAPLMLDGYLFKPAGHKPYPAVVMFHGCAGAISVKTGRITSRFTNMAQLLNDMGYAVLIVDSFNPRGVPQICTVPLADRDIKNRHRVMDAYSALQYLNSRPDIVAGKIGAIGFSHGGSGALSSMDATTEAYGRSEQKFAASVALYPGCGWQSRKQPEFSAYGPLLILAGEKDDWTPVEPCRKLAARSQQRNEPVELVVYPDAYHAFDSTSEVYVRKDVPNGVNGSAGVHVGANPAAREAAYKRIREFFHQHLRP

SEQ ID NO: 3 - protein, 23 aa, Pusillimonas noertemannii
KDVPNGVNGSAGVHVGANPAARE

SEQ ID NO: 4 - protein, 18 aa, Pusillimonas noertemannii
KFAASVALYPGCGWQSRK

SEQ ID NO: 5 - protein, 26 aa, Pusillimonas noertemannii
KIGAIGFSHGGSGALSSMDATTEAYG

SEQ ID NO: 6 - protein, 24 aa, Pusillimonas noertemannii
RKDVPNGVNGSAGVHVGANPAARE

(19) United States
(12) Patent Application Publication
(10) Pub. No.: US 2016/0115466 A1
(43) Pub. Date: Apr. 28, 2016

(54) ENZYME AND USES THEREOF

(71) Applicant: UCL BUSINESS PLC, Greater London (GB)

(72) Inventors: Peter Taylor, Billingshurst (GB); David Negus, London (GB)

(73) Assignee: UCL BUSINESS PLC, London (GB)

(21) Appl. No.: 14/892,881

(22) PCT Filed: May 16, 2014
(86) PCT No.: PCT/GB2014/051520

§ 371 Date: Nov. 20, 2015

(30) Foreign Application Priority Data
May 21, 2013 (GB) 1309178.0

(51) Int. Cl. C12N 9/48; A61K 31/496; C07K 16/40; A61K 38/48

(52) U.S. Cl. CPC C12N 9/485; A61K 38/4813; A61K 31/496; C07K 16/40; C12Y 304/19

(57) ABSTRACT

The present invention relates generally to methods and materials relating to newly characterised enzymes from Pusillimonas noertemannii, or variants thereof, capable degrading poly-γ-D-glutamic acid, for example as is present in the capsule of B. anthracis. Such depolymerases have utility as therapeutics.

### TECHNICAL FIELD

The present invention relates generally to methods and materials relating to newly characterised enzymes capable degrading poly-γ-D-glutamic acid, for example as is present in the capsule of B. anthracis.

### BACKGROUND ART

Experimental infections due to bacteria expressing a polysaccharide or polypeptide capsule may be resolved by administration of depolymerases that selectively hydrolyse the external protective layer.

Naturally occurring anthrax is acquired following contact with infected animals or animal products contaminated with the encapsulated, spore-forming Gram-positive rod Bacillus anthracis.¹ Inhalation anthrax, the most severe form of the disease, is usually fatal.

Natural infections are rarely encountered in the developed world, but the potential for aerosol-mediated spread of B. anthracis spores by rogue states and terrorists is of growing concern. Countermeasures to this threat include vaccination, quarantine and chemotherapy and to this end a limited number of antibiotics have been identified for prophylaxis and treatment.^{2,3} As there have been no clinical studies of the treatment of anthrax in humans, recommended antibiotic regimens are based on empirical treatments for sepsis.³ Most naturally occurring anthrax strains are sensitive to penicillin, which is approved by the FDA for this indication, as is doxycycline. Likewise, ciprofloxacin has been identified as a suitable alternative due to its efficacy in animal models of anthrax but has not been studied in human anthrax.³

B. anthracis causes lethal infection due to the elaboration by the vegetative bacillary form of a protein exotoxin complex and a capsule composed of poly-γ-D-glutamic acid (PDGA).^{1,7} The vital role of the anti-phagocytic PDGA capsule has been unambiguously established: mutants deleted for the capBCAD capsule biosynthetic operon are fully attenuated in a murine model of inhalation anthrax.⁸ In inhalation anthrax, endospores gain access to the alveolar spaces where they are phagocytosed by macrophages prior to germination.¹ Macrophages containing bacilli detach and migrate to regional lymph nodes, creating regional haemorrhagic lymphadenitis followed by spread through the blood and lymph. Capsule expression occurs soon after spore germination in response to host signals that include raised CO₂,⁷ before migration to lymph nodes,⁹ and ensures extracellular bacterial replication. These events indicate that progression of the infection could be interrupted by removal of the anti-phagocytic PDGA capsule during the early stages of the systemic disease.

Earlier studies with depolymerases that remove the protective polysaccharide capsule from neuropathogenic strains of Escherichia coli during experimental infections have highlighted the therapeutic potential of this approach.¹⁰⁻¹³ Anthrax is an ideal candidate for this therapeutic paradigm: the infection is caused by a single, phylogenetically homogeneous bacterial species and all strains elaborate a unique polypeptide capsule that is essential for pathogenesis. Thus, “capsule stripping” has the potential to deliver an exquisitely selective therapeutic agent that would confound attempts, through the introduction of multiple antibiotic resistance genes into B. anthracis, to circumvent current therapeutic strategies.

Publication US2010/0226906 relates to isolated, recombinant CapD and recombinant PghP for use in digesting capsule comprising polyglutamate polymers and for treatment of infections caused by bacilli having a polyglutamate capsule, such as anthrax

Notwithstanding the above disclosures, it can be seen that new agents to treat the disease in civilian and animal populations, to protect and treat military personnel who may be exposed on the battlefield, and to counter potential terrorist outrages would provide a contribution to the art.

### DISCLOSURE OF THE INVENTION

The inventors have characterised a novel Poly-γ-D-glutamic acid depolymerase from Pusillimonas noertemannii.

The enzyme is has a molecular weight of 31.4 kDa and a pl 8.81. Both His₆-tagged and non-tagged versions of EnvD have been expressed and recovered from the pET26b (+) E. coli BL21 3ED vector expression system.

As explained in the Examples below, the identification of the enzyme (termed herein “EnvD”) was not straightforward, and initial attempts to identify the enzyme from gel spots (by LC/MS-MS) were unsuccessful. Furthermore the amino acid sequence of the enzyme does not share a high degree of homology with previously characterised glutamic acid depolymerases.

This enzyme has utility as novel therapeutic agent for use against anthrax infection. The disclosure thus provides for inter alia novel nucleic acids encoding EnvD or variants thereof, as well as EnvD polypeptides, compositions comprising them, and uses of the same in methods of treatment or prophylaxis e.g. against anthrax.

Some aspects of the invention will now be discussed in more detail:

### Nucleic Acids of the Invention

Thus in one aspect of the present invention there is disclosed an isolated nucleic acid molecules encoding a Poly-γ-D-glutamic acid (PGDA) depolymerase from Pusillimonas noertemannii.

In particular embodiments the invention provides an isolated PGDA depolymerase gene having a sequence provided herein as SEQ. ID. 1, or variants thereof as discussed below.

Nucleic acid molecules according to the present invention may be provided isolated and/or purified from their natural environment, in substantially pure or homogeneous form, or free or substantially free of other nucleic acids of the species of origin. Where used herein, the term “isolated” encompasses all of these possibilities.

The nucleic acid molecules may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

Preferred nucleic acids consist or consist essentially of the nucleotide sequence in question, optionally in an expression vector as described in more detail below.

Nucleic acid according to the present invention may include cDNA, RNA, genomic DNA and modified nucleic acids or nucleic acid analogs. Where a DNA sequence is specified, e.g. with reference to a figure, unless context requires otherwise the RNA equivalent, with U substituted for T where it occurs, is encompassed. Where a nucleic acid of the invention is referred to herein, the complement of that nucleic acid will also be embraced by the invention.

The ‘complement’ of a given nucleic acid (sequence) is the same length as that nucleic acid (sequence), but is 100% complementary thereto.

Where genomic nucleic acid sequences of the invention are disclosed, nucleic acids comprising any one or more (e.g. 2) introns or exons from any of those sequences are also embraced.

A PGDA depolymerase gene in this context is one which encodes a polypeptide capable of hydrolysing PGDA, which can be readily assessed by those skilled in the art using the assays known in the art or described herein.

A ‘coding sequence’ or ‘encoding sequence’ is a polynucleotide sequence which is transcribed into mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include but is not limited to mRNA, DNA (including cDNA), and recombinant polynucleotide sequences.

The term ‘polypeptide’ refers to a polymer of amino acids and does not refer to a specific length of the product; thus, peptides, oligopeptides, and proteins are included within the definition of polypeptide. This term also does not refer to or exclude post-expression modifications of the polypeptide, for example, glycosylations, acetylations, phosphorylations and the like. Included within the definition are, for example, polypeptides containing one or more analogues of an amino acid (including, for example, unnatural amino acids, PNA, etc.), polypeptides with substituted linkages, as well as other modifications known in the art, both naturally occurring and non-naturally occurring. Some of these variants are described in more detail below.

A nucleic acid of the present invention may encode the amino acid sequence described herein as SEQ ID No 2 (EnvD) e.g. be degeneratively equivalent to SEQ ID No 1.

### Nucleic Acid Variants

In a further aspect of the present invention there are disclosed nucleic acids which are variants of the sequences of the first aspect.

A variant nucleic acid molecule shares homology with, or is identical to, all or part of the coding sequence discussed above. Generally, variants may encode, or be used to isolate or amplify nucleic acids which encode, polypeptides which are PGDA depolymerases and/or which will specifically bind to an antibody raised against the PGDA depolymerase of SEQ ID No 1.

The EnvD polypeptide antigen used in the present invention is preferably a full-length protein as described herein. However variants are also contemplated e.g. a substantially full-length version, i.e. containing functional fragments thereof (e.g. fragments which are not missing sequence essential to the formation or retention of enzyme activity)

Polypeptide variants of the present invention can be encoded by artificial nucleic acids (i.e. containing sequences which have not originated naturally) which can be prepared by the skilled person in the light of the present disclosure. Alternatively they may be novel, naturally occurring, nucleic acids, which have been or may be isolatable using the sequences of the present invention e.g. from other soil bacteria.

Thus a variant may be a distinctive part or fragment (however produced) corresponding to a portion of the sequence provided.

The fragments may encode particular functional parts of the polypeptide. Equally the fragments may have utility in probing for, or amplifying, the sequence provided or closely related ones. Suitable lengths of fragment, and conditions, for such processes are discussed in more detail below.

Also included are nucleic acids which have been extended at the 3′ or 5′ terminus. Sequence variants which occur naturally may include alleles or other homologues (which may include polymorphisms or mutations at one or more bases).

Artificial variants (derivatives) may be prepared by those skilled in the art, for instance by site directed or random mutagenesis, or by direct synthesis. Preferably the variant nucleic acid is generated either directly or indirectly (e.g. via one or amplification or replication steps) from an original nucleic acid having all or part of the sequences of the first aspect.

The term “variant” nucleic acid as used herein encompasses all of these possibilities. When used in the context of polypeptides or proteins it indicates the encoded expression product of the variant nucleic acid.

More generally homology (i.e. similarity or identity) may be as defined using sequence comparisons are made using BestFit and GAP programs of GCG, Wisconsin Package 10.0 from the Genetics Computer Group, Madison, Wis. CLUSTAL is also a matrix used by BestFit. Parameters are preferably set, using the default settings, as follows: Gap Creation pen: 9; Gapext pen: 2. Homology may be at the nucleotide sequence and/or encoded amino acid sequence level. Preferably, the nucleic acid and/or amino acid sequence shares at least about 50%, or 60%, or 70%, or 80% homology, most preferably at least about 81%, 82%, 83%, 84%, 85%, 86%, 87%, 88%, 89%, 90%, 91%, 92%, 93%, 94%, 95%, 96%, 97%, 98% or 99% homology with SEQ. ID. 1.

In particular the invention provides an isolated PGDA depolymerase gene having a sequence which is at least about 60% homologous to the nucleic acid sequence provided herein as SEQ. ID. 1.

It further provides an isolated polypeptide having an amino acid sequence which is at least 60% homologous to the amino acid sequence provided herein as SEQ. ID. 2.

Thus a variant polypeptide in accordance with the present invention may include within the sequences shown herein, a single amino acid or 2, 3, 4, 5, 6, 7, 8, or 9 changes, about 10, 15, 20, 30, 40 or 50 changes, or greater than about 50, 60, 70, 80, 90, 100 or 150 changes. In addition to one or more changes within the amino acid sequence shown, a variant polypeptide may include additional amino acids at the C-terminus and/or N-terminus.

Thus in a further aspect of the invention there is disclosed a method of producing a derivative nucleic acid comprising the step of modifying the coding sequence of a nucleic acid of the present invention e.g. SEQ. ID. 1.

Changes to a sequence, to produce a derivative, may be by one or more of addition, insertion, deletion or substitution of one or more nucleotides in the nucleic acid, leading to the addition, insertion, deletion or substitution of one or more amino acids in the encoded polypeptide. Changes may be desirable for a number of reasons, including introducing or removing the following features: restriction endonuclease sequences; codon usage; other sites which are required for post translation modification; cleavage sites in the encoded polypeptide; motifs in the encoded polypeptide (e.g. binding sites). Leader or other targeting sequences may be added or removed from the expressed protein to determine its location following expression. All of these may assist in efficiently cloning and expressing an active polypeptide in recombinant form (as described below).

The inventors have expressed EnvD in E. coli. In common with many recombinant proteins expressed in E. coli,²³ they recovered both bioactive soluble and inactive insoluble fractions from induced cultures.

It will be appreciated that solubility of recombinant EnvD may be improved by those skilled in the art in the light of the present disclosure by reference to the primary amino acid sequence described herein and by systematic determination of optimum conditions for soluble protein expression; variables such as culture temperature, IPTG concentration and time allowed for expression contribute to the correct folding of recombinant proteins.^{24,26} DiANNA²⁶ and SignalP²⁷ software indicate the presence of two disulfide bonds and a highly hydrophobic N-terminal signal peptide at positions 1-18 of SEQ ID No 2. Over-expression of proteins in the reducing environment of the cytoplasm strongly disfavours the formation of stable disulfide bonds.²⁸ To improve EnvD solubility, the signal peptide can be removed using PCR (see below) and protein expressed in E. coli Origami, which possesses mutations in thioredoxin reductase and glutathione reductase genes that greatly enhance disulfide bond formation in the cytoplasm.²⁸ Identification of conserved functional domains using the NCBI conserved domain search tool provides an opportunity to amplify regions that are essential for depolymerase activity and remove hydrophobic sequences that do not contribute to catalytic activity; expression of a truncated protein with a more hydrophilic nature will further enhance protein solubility.

Alternatively EnvD polypeptides may be expressed as fusion proteins, with fusion partners selected from those known in the art to facilitate recovery or folding—see e.g. Ahn J-H, Keum J-W, Kim D-M (2011) Expression Screening of Fusion Partners from an E. coli Genome for Soluble Expression of Recombinant Proteins in a Cell-Free Protein Synthesis System. PLoS ONE 6(11): e26875. doi:10.1371/journal.pone.0026875. The fusion partner subsequently be removed.

Other desirable mutation may be random or site directed mutagenesis in order to alter the activity (e.g. specificity) or stability of the encoded polypeptide. The inventors have shown that the active site residues are likely at positions 150, 208 and 240 of SEQ ID No 2. Changes may be by way of conservative variation, i.e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation.

Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e.g. altered stability or specificity.

In a further aspect of the present invention there is provided a method of identifying and/or cloning a nucleic acid variant from a bacterium e.g. a soil isolated bacterium or fungus e.g. a Pusillimonas spp. e.g. Pusillimonas noertemannii which method employs a distinctive EnvD PGDA depolymerase nucleotide sequence (e.g. as present in SEQ. ID. 1 or the complement thereof, or degenerate primers based thereon).

An oligonucleotide for use in probing or amplification reactions comprise or consist of about 30 or fewer nucleotides in length (e.g. 18, 21 or 24). Generally specific primers are upwards of 14 nucleotides in length. For optimum specificity and cost effectiveness, primers of 16-24 nucleotides in length may be preferred. Those skilled in the art are well versed in the design of primers for use processes such as PCR. If required, probing can be done with entire restriction fragments of the gene disclosed herein which may be 100's or even 1000's of nucleotides in length.

Preferably the probe/primer is distinctive in the sense that it is present in SEQ ID No 1 but not in PGDA depolymerase gene sequences of the prior art.

In a further embodiment, a variant in accordance with the present invention is also obtainable by means of a method which includes:

(a) providing a preparation of nucleic acid, e.g. from bacteria,
(b) providing a nucleic acid molecule which is a probe as described above,
(c) contacting nucleic acid in said preparation with said nucleic acid molecule under conditions for hybridisation of said nucleic acid molecule to any said gene or homologue in said preparation, and identifying said gene or homologue if present by its hybridisation with said nucleic acid molecule.

Probing may employ the standard Southern blotting technique. For instance DNA may be extracted from cells and digested with different restriction enzymes. Restriction fragments may then be separated by electrophoresis on an agarose gel, before denaturation and transfer to a nitrocellulose filter or nylon. Labelled probe may be hybridised to the DNA fragments on the filter and binding determined. DNA for probing may be prepared from RNA preparations from cells.

Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as is described hereinafter. Probing may optionally be done by means of so-called “nucleic acid chips” (see Marshall & Hodgson (1998) Nature Biotechnology 16: 27-31, for a review).

Preliminary experiments may be performed by hybridising under low stringency conditions. For probing, preferred conditions are those which are stringent enough for there to be a simple pattern with a small number of hybridisations identified as positive which can be investigated further.

For instance, screening may initially be carried out under conditions, which comprise a temperature of about 37° C. or less, a formamide concentration of less than about 50%, and a moderate to low salt (e.g. Standard Saline Citrate (“SSC”)=0.15 M sodium chloride; 0.15 M sodium citrate; pH 7) concentration.

Alternatively, a temperature of about 50° C. or less and a high salt (e.g. “SSPE” 0.180 mM sodium chloride; 9 mM disodium hydrogen phosphate; 9 mM sodium dihydrogen phosphate; 1 mM sodium EDTA; pH 7.4). Preferably the screening is carried out at about 37° C., a formamide concentration of about 20%, and a salt concentration of about 5×SSC, or a temperature of about 50° C. and a salt concentration of about 2×SSPE. These conditions will allow the identification of sequences which have a substantial degree of homology (similarity, identity) with the probe sequence, without requiring the perfect homology for the identification of a stable hybrid.

Suitable conditions include, e.g. for detection of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

It is well known in the art to increase stringency of hybridisation gradually until only a few positive clones remain. Suitable conditions would be achieved when a large number of hybridising fragments were obtained while the background hybridisation was low.

Using these conditions nucleic acid libraries, e.g. cDNA libraries representative of expressed sequences, may be searched. Those skilled in the art are well able to employ suitable conditions of the desired stringency for selective hybridisation, taking into account factors such as oligonucleotide length and base composition, temperature and so on. One common formula for calculating the stringency conditions required to achieve hybridization between nucleic acid molecules of a specified sequence homology is (Sambrook et al., 1989): Tₘ=81.5° C.+16.6 Log [Na+]+0.41 (% G+C)−0.63 (% formamide)−600/#bp in duplex. As an illustration of the above formula, using [Na+]=[0.368] and 50-% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. Such a sequence would be considered substantially homologous to the nucleic acid sequence of the present invention.

Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below) or RN′ase cleavage. The identification of successful hybridisation is followed by isolation of the nucleic acid which has hybridised, which may involve one or more steps of PCR or amplification of a vector in a suitable host.

Thus one embodiment of this aspect of the present invention is nucleic acid including or consisting essentially of a sequence of nucleotides complementary to a nucleotide sequence hybridisable with any encoding sequence provided herein. Another way of looking at this would be for nucleic acid according to this aspect to be hybridisable with a nucleotide sequence complementary to any encoding sequence provided herein. Of course, DNA is generally double-stranded and blotting techniques such as Southern hybridisation are often performed following separation of the strands without a distinction being drawn between which of the strands is hybridising.

In a further embodiment, hybridisation of nucleic acid molecule to a variant may be determined or identified indirectly, e.g. using a nucleic acid amplification reaction, particularly the polymerase chain reaction (PCR)(see “PCR protocols; A Guide to Methods and Applications”, Eds. Innis et al, Academic Press, New York, (1990)).

As used herein, unless the context demands otherwise, the term “Env D nucleic acid” or similar is intended to cover any of the nucleic acids of the invention described above, including functional variants.

Likewise, as used herein, unless the context demands otherwise, the term “Env D polypeptide” or “Env D based polypeptide” or “Env D derived polypeptide” or similar, is intended to any of the polypeptides of the invention described above, including functional variants of SEQ ID No 2.

### Vectors and Hosts

In one aspect of the present invention, the Env D nucleic acid described above is in the form of a recombinant and preferably replicable vector.

“Vector” is defined to include, inter alia, any plasmid, cosmid, phage or other vector in double or single stranded linear or circular form which may or may not be self-transmissible or mobilizable, and which can transform prokaryotic or eukaryotic host either by integration into the cellular genome or exist extrachromosomally (e.g. autonomous replicating plasmid with an origin of replication).

A vector including nucleic acid according to the present invention need not include a promoter or other regulatory sequence, particularly if the vector is to be used to introduce the nucleic acid into cells for recombination into the genome.

Preferably the nucleic acid in the vector is under the control of, and operably linked to, an appropriate promoter or other regulatory elements for transcription in a host cell such as a microbial, e.g. bacterial cell. In the case of genomic DNA, this may contain its own promoter or other regulatory elements and in the case of cDNA this may be under the control of an appropriate promoter or other regulatory elements for expression in the host cell.

Example vectors for use in the present invention include e.g. both his₆-tagged and non-tagged variants (e.g. the pET26b vector) which can be used, for example, in E. coli BL21.

By “promoter” is meant a sequence of nucleotides from which transcription may be initiated of DNA operably linked downstream (i.e. in the 3′ direction on the sense strand of double-stranded DNA). “Operably linked” means joined as part of the same nucleic acid molecule, suitably positioned and oriented for transcription to be initiated from the promoter. DNA operably linked to a promoter is “under transcriptional initiation regulation” of the promoter.

Thus this aspect of the invention provides a gene construct, preferably a replicable vector, comprising a promoter operatively linked to a nucleotide sequence provided by the present invention, such as SEQ. ID. 1 or a variant thereof.

Generally speaking, those skilled in the art are well able to construct vectors and design protocols for recombinant gene expression. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press (or later editions of this work).

Many known techniques and protocols for manipulation of nucleic acid, for example in preparation of nucleic acid constructs, mutagenesis (see above discussion in respect of variants), sequencing, introduction of DNA into cells and gene expression, and analysis of proteins, are described in detail in Current Protocols in Molecular Biology, Second Edition, Ausubel et al. eds., John Wiley & Sons, 1992. The disclosures of Sambrook et al. and Ausubel et al. are incorporated herein by reference.

In one embodiment of this aspect of the present invention, there is provided a gene construct, preferably a replicable vector, comprising an inducible promoter operatively linked to a nucleotide sequence provided by the present invention. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

The present invention also provides methods comprising introduction of such a construct into a host cell.

The host may be, for example, a lower eukaryote, a prokaryote, or a higher eukaryote.

The term ‘lower eukaryote’ refers to host cells such as yeast, fungi and the like. Lower eukaryotes are generally (but not necessarily) unicellular. Preferred lower eukaryotes are yeasts, particularly species within Saccharomyces, Schizosaccharomyces, Kluveromyces, Pichia (e.g. Pichia pastoris), Hansenula (e.g. Hansenula polymorpha, Yarowia, Schwaniomyces, Schizosaccharomyces, Zygosaccharomyces and the like. Saccharomyces cerevisiae, S. carlsbergensis and K. lactis are the most commonly used yeast hosts, and are convenient fungal hosts.

The term ‘prokaryotes’ refers to hosts such as E. coli, Lactobacillus, Lactococcus, Salmonella, Streptococcus, Bacillus subtilis or Streptomyces. All of these hosts are contemplated within the present invention.

The term ‘higher eukaryote’ refers to host cells derived from higher animals, such as mammals, reptiles, insects, and the like. Presently preferred higher eukaryote host cells are derived from Chinese hamster (e.g. CHO), monkey (e.g. COS and Vero cells), baby hamster kidney (BHK), pig kidney (PK15), rabbit kidney 13 cells (RK13), the human osteosarcoma cell line 143 B, the human cell line HeLa and human hepatoma cell lines like Hep G2, and insect cell lines (e.g. Spodoptera frugiperda). The host cells may be provided in suspension or flask cultures, tissue cultures, organ cultures and the like. Alternatively the host cells may also be transgenic animals.

Preferred hosts are microbial e.g. lower eukaryote or prokaryote.

Where used herein the term ‘recombinant host cells’, ‘host cells’, ‘cells’, ‘cell lines’, ‘cell cultures’, and other such terms denote microorganisms or higher eukaryotic cell lines cultured as unicellular entities refer to cells which can be or have been, used as recipients for a recombinant vector or other transfer polynucleotide, and include the progeny of the original cell which has been transfected. It is understood that the progeny of a single parental cell may not necessarily be completely identical in morphology or in genomic or total DNA complement as the original parent, due to natural, accidental, or deliberate mutation.

In a further aspect of the invention, there is disclosed a recombinant host cell containing a heterologous construct according to the present invention, especially a microbial cell.

The term “heterologous” is used broadly in this aspect to indicate that the gene/sequence of nucleotides in question (i.e. the PGDA depolymerase gene) have been introduced into said cell or an ancestor thereof, using genetic engineering, i.e. by human intervention.

Nucleic acid heterologous to a host cell may be non-naturally occurring in cells of that type, variety or species. A further possibility is for a nucleic acid sequence to be placed within a host cell in which it or a homolog is found naturally, but wherein the nucleic acid sequence is linked and/or adjacent to nucleic acid which does not occur naturally within the cell, or cells of that type or species, such as operably linked to one or more regulatory sequences, such as a promoter sequence, for control of expression.

The host cell is preferably transformed by the construct, which is to say that the construct becomes established within the cell, altering one or more of the cell's characteristics and hence phenotype e.g. with respect to PDGA hydrolysis.

The invention further encompasses a host cell transformed with nucleic acid or a vector according to the present invention especially a microbial cell. In the transformed cell the heterologous gene may be on an extra-genomic vector or incorporated, preferably stably, into the genome. There may be more than one heterologous nucleotide sequence per haploid genome.

### Other Methods and Aspects of the Invention

The invention further provides a method of influencing or affecting the PDGA (especially high molecular weight PDGA) hydrolysing properties of a cell, the method including the step of causing or allowing expression of a heterologous nucleic acid sequence as discussed above within the cell. The method may be followed by the step of assessing the PDGA hydrolysing properties e.g. using assays known in the art or described herein.

The step may be preceded by the earlier step of introduction of the nucleic acid into a cell or an ancestor thereof.

Following expression, the recombinant product may, if required, be isolated from the expression system, if necessary (i.e. if they are not secreted) by lysing the host cell.

Thus the invention provides a method for producing and purifying recombinant EnvD polypeptide comprising:

(i) culturing a host cell containing a vector expressing an EnvD polypeptide in a suitable culture medium,
(ii) causing expression of said vector sequence as defined above under suitable conditions for production of soluble protein and optionally,
(iii) lysing said transformed host cells and recovering said EnvD polypeptide, or recovering secreted EnvD polypeptide from the culture medium.

As important aspect of the present invention is therefore the expression product of any of the PGDA depolymerase nucleic acid sequences disclosed above, plus also methods of making the recombinant purified expression product by expression from encoding nucleic acid therefore under suitable conditions, which may be in suitable host cells.

The invention provides compositions comprising said recombinant EnvD polypeptide.

The term ‘recombinantly expressed’ used within the context of the present invention refers to the fact that the proteins of the present invention are produced by expression from a heterologous gene e.g. in prokaryotes, or lower or higher eukaryotes as discussed in detail above.

The term ‘purified’ as applied to proteins herein refers to a composition wherein the desired protein comprises at least 35% of the total protein component in the composition. The desired protein preferably comprises at least 40%, more preferably at least about 50%, more preferably at least about 60%, still more preferably at least about 70%, even more preferably at least about 80%, even more preferably at least about 90%, and most preferably at least about 95% of the total protein component. The composition may contain other compounds such as carbohydrates, salts, lipids, solvents, and the like, without affecting the determination of the percentage purity as used herein.

The term ‘essentially purified proteins’ refers to proteins purified such that they can be used for in vitro diagnostic methods and as a prophylactic compound. These proteins are substantially free from cellular proteins, vector-derived proteins or other components. The proteins of the present invention are purified to homogeneity, at least 80% pure, preferably, 90%, more preferably 95%, more preferably 97%, more preferably 98%, more preferably 99%, even more preferably 99.5%.

A preferred polypeptide includes the amino acid sequence shown in SEQ. ID. 2. However a polypeptide according to the present invention may be a variant (allele, fragment, derivative, mutant or homologue etc.) of these polypeptides, as described herein.

### Therapeutic and Prophylactic Utilities

Recombinant EnvD polypeptide can be used as a therapeutic or prophylactic for infections by organisms producing a PGDA.

In one embodiment the EnvD polypeptides of the present invention may be used in a method to remove the capsule from the surface of a bacterium e.g. a pathogen e.g. a bacillus e.g. B. anthracis.

This may have the effect of rendering the bacteria susceptible to neutrophil killing. For example the method may have the purpose of enabling phagocytes to ingest and kill bacilli by removing the capsule from the surface of the organism.

The invention provides for use of the EnvD polypeptides described herein the therapy of a variety of illnesses caused by organisms producing a capsule comprising PGDA, comprising administering a composition comprising EnvD polypeptide to a subject in need of the same.

The invention also provides a therapeutic composition for treatment of prophylaxis of anthrax infection comprising EnvD polypeptide. The therapy may also include conventional antibiotics.

In one aspect of the invention there is provided a method for inhibiting replication of B. anthracis in a subject, said method comprising the steps of administering to subject a composition comprising an EnvD polypeptide of the invention.

In one aspect of the invention there is provided a method of inhibiting the virulence or growth, or reducing the number, of B. anthracis in a subject, said method comprising the steps of administering to subject a composition comprising an EnvD polypeptide of the invention.

In one aspect there is a provided use of a composition comprising an EnvD polypeptide of the invention in the preparation of an agent for the treatment and/or prophylaxis of B. anthracis in a subject. Said treatment may comprise any of the methods described herein, particularly those methods which are therapeutic methods practiced on the animal body.

In one aspect there is a provided a composition comprising an EnvD polypeptide of the invention for use in any of the methods described herein, particularly those methods which are therapeutic methods practiced on the animal body.

In these aspects the EnvD polypeptide may be administered as an ‘effective amount’. The term ‘effective amount’ for a therapeutic or prophylactic treatment refers to an amount of epitope-bearing polypeptide sufficient to facilitate clearance of polyglutamate capsule-producing bacteria from the individual to which it is administered. Preferably, the effective amount is sufficient to effect treatment, as defined above. The exact amount necessary will vary according to the application. For therapeutic applications, for example, the effective amount may vary depending on the species, age, and general condition of the individual, the severity of the condition being treated, the particular polypeptide selected and its mode of administration, etc. It is also believed that effective amounts will be found within a relatively large, non-critical range. An appropriate effective amount can be readily determined using only routine experimentation. Preferred ranges of EnvD polypeptide for prophylaxis are about 0.01 to 100,000 ug/dose, more preferably about 0.1 to 10,000 ug/dose, most preferably about 10-500 ug/dose. Several doses may be needed per individual in order to achieve resolution of infection. It should be noted that the term “prophylaxis” or the like as used herein does not circumscribe complete success, but rather indicates that the treatment is given in advance of possible exposure or symptoms related to the disease in question with the purpose of reducing the impact of the disease on an individual.

Treatment of individuals having an infection comprises administering a therapeutic composition in a sufficient amount, possibly accompanied by pharmaceutically acceptable carrier, or other drugs known to promote clearing of the infections, e.g. antibiotics, in order to produce a reduction in symptoms of the infection. In general, this will comprise administering a therapeutically or prophylactically effective amount of the EnvD polypeptide of the present invention to a susceptible subject or one exhibiting infection symptoms.

In providing a patient with EnvD polypeptide, the dosage of administered agent will vary depending upon such factors as the patient's age, weight, height, sex, general medical condition, previous medical history, etc.

The enzymes capable of degrading the capsule are intended to be provided to recipient subjects in an amount sufficient to effect a reduction in infection symptoms. An amount is said to be sufficient to “effect” the reduction of infection symptoms if the dosage, route of administration, etc. of the agent are sufficient to influence such a response. Responses to antibody administration can be measured by analysis of subject's vital signs.

Administration of the therapy could be performed orally or parenterally, or intravenously in amounts sufficient to enable the enzymes to degrade the organism's capsule. Parenteral forms are preferred and these are discussed in more detail herein.

### Compositions and Dosage Forms

The EnvD-based polypeptides of the present invention can be formulated in dosage forms using methods well known to those skilled in the art.

For example high throughput stability and aggregation studies can be conducted at different pH, salt and protein/excipient concentrations to confirm optimal conditions to formulate EnvD. Microfluidic determination of EnvD stability and ligand affinity by assessing the thermodynamics of reversible protein precipitation using NaCl and NH₄SO₄ in microplate checkerboard format may also be performed. Conditions that promote correct protein refolding can then be employed together with pH and salt concentration data to define a solution that will promote active monomeric protein to facilitate administration to infected mice of a range of depolymerase concentrations.

Such physicochemical characterisation and pre-formulation is available commercially e.g. at the EPSRC Centre for Innovative Manufacturing in Emergent Macromolecular Therapies, based at UCL.

Compositions or dosage forms of the present invention may e.g. be provided as a freeze-dried (lyophilised) composition or a solution formulation.

A freeze-dried form of EnvD would be expected to be more easily stored and a ready-to-use liquid form would be expected to be more convenient with respect to administration.

Therapeutic compositions can be prepared according to methods known in the art—see for example US20100226906. The present compositions comprise an amount of a recombinant EnvD polypeptide as defined herein, usually combined with a pharmaceutically acceptable carrier.

Pharmaceutically acceptable carriers include any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers; and inactive virus particles. Such carriers are well known to those of ordinary skill in the art. The carrier may be comprised of a saline solution, dextrose, albumin, a serum, or any combinations thereof.

The compositions typically will contain pharmaceutically acceptable vehicles, such as water, saline, glycerol, ethanol, etc. Additionally, auxiliary substances, such as wetting or emulsifying agents, pH buffering substances, preservatives, and the like, may be included in such vehicles.

Typically, the compositions are prepared as injectables, either as liquid solutions or suspensions; solid forms suitable for solution in, or suspension in, liquid vehicles prior to injection may also be prepared. The preparation also may be emulsified or encapsulated in liposomes. Solutions for infusion or injection may be prepared in a conventional manner, e.g. with the addition of preservatives such as p-hydroxybenzoates or stabilizers such as alkali metal salts of ethylenediamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein.

In cases where intramuscular injection is the mode of administration, an isotonic formulation can be used. Generally, additives for isotonicity can include sodium chloride, dextrose, mannitol, sorbitol and lactose. In some cases isotonic solutions such as phosphate buffered saline are preferred. Stabilizers include gelatin and albumin which may be included in the formulation. In some embodiments, a vasoconstriction agent is added to the formulation. The pharmaceutical preparations according to the present invention are provided sterile and pyrogen free.

The polypeptides of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A. ed., Mack Easton Pa. (1980)). In order to form a pharmaceutically acceptable composition suitable for effective administration, such compositions will contain an effective amount of the above-described compounds together with a suitable amount of carrier vehicle.

Additional pharmaceutical methods may be employed to control the duration of action. Control release preparations may be achieved through the use of polymers to complex or absorb the compounds. The controlled delivery may be exercised by selecting appropriate macromolecules (for example polyesters, polyamino acids, polyvinyl, pyrrolidone, ethylenevinylacetate, methylcellulose, carboxymethylcellulose, or protamine sulfate) and the concentration of macromolecules as well as the method of incorporation in order to control release. Another possible method to control the duration of action by controlled release preparations is to incorporate the compounds of the present invention into particles of a polymeric material such as polyesters, polyamino acids, hydrogels, polylactic acid or ethylene vinylacetate copolymers. Alternatively, instead of incorporating these agents into polymeric particles, it is possible to entrap these materials in microcapsules prepared, for example, interfacial polymerization, for example, hydroxymethylcellulose or gelatin-microcapsules and poly(methylmethacylate)-microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1980).

Administration of the compounds disclosed herein may be carried out by any suitable means, including parenteral injection (such as intravenous intraperitoneal, subcutaneous, or intramuscular injection), in ovo injection of birds, orally, or by topical application of the enzymes (typically carried in a pharmaceutical formulation) to an airway surface. Topical application to an airway surface can be carried out by intranasal administration (e.g., by use of dropper, swab, or inhaler which deposits a pharmaceutical formulation intranasally). Topical application to an airway surface can also be carried out by inhalation administration, such as by creating respirable particles of a pharmaceutical formulation (including both solid particles and liquid particles) containing the antibodies as an aerosol suspension, and then causing the subject to inhale the respirable particles. Methods and apparatus for administering respirable particles of pharmaceutical formulations are well known, and any conventional technique can be employed. Oral administration may be in the form of an ingestable liquid or solid formulation. However oral administration is the least preferred method.

In order to accelerate treatment of the infection, the therapeutic agent may further include at least one complementary agent which can also potentiate the bactericidal activity of the enzyme. The complementary agent can be penicillin, ciprofloxacin (used to treat anthrax infection) or any other appropriate antiobiotic.

The present invention also provides kits which are useful for carrying out the present invention. The present kits comprise a first container means containing the enzymes of the invention. The kit also comprises other container means containing solutions necessary or convenient for carrying out the invention. The container means can be made of glass, plastic or foil and can be a vial, bottle, pouch, tube, bag, etc. The kit may also contain written information, such as procedures for carrying out the present invention or analytical information, such as the amount of reagent contained in the first container means. The container means may be in another container means, e.g. a box or a bag, along with the written information.

### Confirming Activity

The efficacy of composition of the present invention e.g. including the EnvD-based polypeptides described herein can be confirmed in known models of inhalation anthrax e.g. murine models. An exemplary assay is provided in the Examples below.

### Other Uses of Polypeptides of the Invention

Purified PGDA depolymerase or variant proteins of the invention, produced recombinantly by expression from encoding nucleic acid therefor, may be used for a variety of purposes e.g. in degrading a PGDA (in vivo or in vitro) or in a diagnostic assay.

In one aspect of the invention there is provided a method for degrading PGDA by providing EnvD polypeptide in an amount sufficient to degrade said polymer. Such methods may be performed in vivo or in vitro.

Purified PGDA depolymerase or variant proteins of the invention may be used to raise antibodies employing techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and might be screened, preferably using binding of antibody to antigen of interest. For instance, Western blotting techniques or immunoprecipitation may be used (Armitage et al, 1992, Nature 357: 80-82). Antibodies may be polyclonal or monoclonal. As an alternative or supplement to immunising a mammal, antibodies with appropriate binding specificity may be obtained from a recombinantly produced library of expressed immunoglobulin variable domains, e.g. using lambda bacteriophage or filamentous bacteriophage which display functional immunoglobulin binding domains on their surfaces; for instance see WO92/01047.

Antibodies raised to a polypeptide or peptide can be used in the identification and/or isolation of homologous polypeptides, and then the encoding genes.

Thus, the present invention provides a method of identifying or isolating a polypeptide with PGDA depolymerase activity (in accordance with embodiments disclosed herein), including screening candidate peptides or polypeptides with a polypeptide including the antigen-binding domain of an antibody (for example whole antibody or a fragment thereof) which is able to bind an PGDA depolymerase peptide, polypeptide or fragment, variant or variant thereof or preferably has binding specificity for such a peptide or polypeptide, such as having an amino acid sequence identified herein.

Specific binding members such as antibodies and polypeptides including antigen binding domains of antibodies (“antibody molecules”) that bind and are preferably specific for the polypeptide of the sequence SEQ. ID. 2 or a mutant, variant or derivative thereof represent further aspects of the present invention, as do their use and methods which employ them.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### Sequence

SEQ ID No 1: nucleotide sequence
>Pusi_1763 Pusi_1763 dienelactone hydrolase 1936140:1937012 forward

ATGGCTTCAGCGGTATTGGCGCTTGTTTTCGCGCTGTCGGGGGTAGCGCA
TGGTCGGGTCGTGTTGCCGGAACCGGTCAATTTTCCTAGCCTGGACAACT
GGAAGGGCGACGCGCCCTTGATGCTGGACGGCTATCTGTTCAAGCCAGCC
GGACACAAGCCCTACCCGGCCGTTGTCATGTTCCACGGTTGTGCGGGCGC
CATATCGGTCAAGACGGGGCGCATAACCTCGCGGTTCACCAATATGGCGC
AGCTGCTGAACGACATGGGTTATGCCGTGCTGATCGTCGACAGCTTCAAT
CCGCGCGGCGTGCCGCAGATCTGCACGGTTCCGTTGGCCGACCGGGACAT
CAAGAACAGGCATCGCGTGATGGATGCCTACAGCGCCTTGCAGTACCTGA
ATTCGCGCCCGGATATCGTTGCGGGCAAGATCGGCGCCATAGGGTTTTCG
CATGGCGGCAGCGGCGCGTTGTCATCAATGGATGCTACAACCGAAGCCTA
TGGCCGTTCGGAGCAGAAGTTTGCGGCTTCGGTGGCACTGTACCCGGGTT
GCGGCTGGCAATCGCGCAAGCAGCCGGAGTTCTCGGCATACGGCCCCCTG
TTGATTCTGGCTGGAGAAAAGGACGATTGGACGCCGGTCGAGCCTTGTCG
AAAGCTGGCGGCGCGCAGCCAGCAGCGTAACGAGCCGGTCGAGTTGGTCG
TGTATCCCGATGCCTATCACGCCTTTGACAGCACCAGCGAGGTCTATGTG
CGCAAAGACGTGCCCAATGGTGTCAATGGTTCGGCAGGCGTGCACGTGGG
GGCCAATCCGGCGGCCCGCGAAGCGGCATACAAACGTATCCGCGAGTTTT
TTCACCAGCATTTGCGTCCGTGA

SEQ ID No 2: amino acid sequence

### >Pusi_1763-1936140:1937012 MW:31473.18

MASAVLALVFALSGVAHGRVVLPEPVNFPSLDNWKGDAPLMLDGYLFKP
AGHKPYPAVVMFHGCAGAISVKTGRITSRFTNMAQLLNDMGYAVLIVDS
FNPRGVPQICTVPLADRDIKNRHRVMDAYSALQYLNSRPDIVAGKIGAI
GFSHGGSGALSSMDATTEAYGRSEQKFAASVALYPGCGWQSRKQPEFSA
YGPLLILAGEKDDWTPVEPCRKLAARSQQRNEPVELVVYPDAYHAFDST
SEVYVRKDVPNGVNGSAGVHVGANPAAREAAYKRIREFFHQHLRP

The conserved S9 active site residues Ser, Asp and His are underlined.

### FIGURES

FIG. 1 Stability of CapD in storage

FIG. 2 Isolation of bacteria with the capacity to metabolize PDGA as a sole source of carbon and energy.

FIG. 3 The mixed culture of isolate 1 and 2 degrades PDGA as shown by (A) viscosity and (B) degradation of PDGA as measured by SDS-PAGE.

FIG. 4 Cellular location of the depolymerase

FIG. 5 (A) Separation of intracellular protein fractions by anion exchange chromatograph. (B) PDGA depolymerase activity of the isolated fractions.

FIG. 6 Stability of EnvD compared to CapD

FIG. 7 1-D and 2-D zymographs showing the location of EnvD

FIG. 8 Identification of P. noertemannii BS8 proteins by LC-MS/MS and selection for cloning

FIG. 9 SDS-PAGE gel showing degradation of PDGA by cloned Pusillimonas genes

FIG. 10 An image of the exact peptides that were matched to the protein using LC/MS-MS

FIG. 11 Analysis of the EnvD amino acid sequence

### EXAMPLES

### General Assays

### Assay for Determination of EnvD Substrate Specificity:

The enzyme can be examined for its capacity to hydrolyse poly-γ-glutamyl polymers and the protein substrates gelatine, bovine serum albumin, fibronectin and the heavily glycosylated human airways mucin MUC5AC.

PDGA substrate is recovered from culture supernatants of Bacillus licheniformis ATCC 9945A grown in Medium E containing 615 μM MnSO₄; the isomeric content of each batch is determined be hplc using a Phenomenex chiral column. We have previously established that this procedure yields polymer predominantly (>95%) in the D isoform and rates of hydrolysis at 37° C. are indistinguishable from those obtained with B. anthracis capsule. We have demonstrated potent PDGA depolymerase activity in 50 mM Tris-HCl and 50 mM NaH₂PO₄ buffers at pH 7.0.

PGA in the L isoform can be purified from culture supernatants of B. licheniformis ATCC 9945A grown in Medium E with low Mn (0.615 μM) content. PGA containing equimolar amounts of L- and D-glutamic acid can be purified from Bacillus subtilis NAFM5. MUC5AC is available from Creative Biomart Inc.

### Assay for Determining Endo- or Exopeptidase Activity

Endo- and exopeptidase activity can be distinguished as follows: the enzyme is incubated at 20° C. and 37° C. with PDGA (˜1 mg), samples removed every 10 min and oligopeptides fractionated using a Superdex 200 molecular sieve³² and by hplc with an Asahipack GF-7 column; commercial α-L-PGA standards are used for column calibratation.³³

### Example 1

### Analysis of CapD

CapD is a γ-glutamyltranspeptidase with the primary function of attachment of PDGA to peptidoglycan; it has also been shown to function as a PDGA depolymerase.¹⁴ CapD is activated following auto-cleavage and the two subunits must remain in close association and bound to the bacterial envelope in order to display enzymatic activity. The capacity of the enzyme to degrade PDGA relies, not on a classical protease mechanism, but on transfer of a γ-glutamyl moiety from PDGA to a nucleophile acceptor such as an amino acid, a peptide or a free amino function by a non-sequential “ping-pong” mechanism. We and others^{14,15} have noted that, whilst recombinant CapD rapidly removes the PDGA capsule from B. anthracis, it is markedly unstable; this is likely to be a reflection of its mode of enzymatic action and stringent requirements for activity. CapD did afford some protection to mice infected with B. anthracis by the intraperitoneal route¹⁶ but attempts to engineer pharmaceutical stability into the enzyme led to a marked reduction in its capsule degrading capacity.¹⁷

More specifically, characterization of the molecular mechanism of PDGA degradation by CapD has emphasized the catalytic requirements for optimal polymer hydrolysis under in vitro conditions and also under more complex and variable in vivo environments. CapD is a two-substrate enzyme that has stringent donor and acceptor substrate preferences that impact significantly on its capacity to hydrolyze the B. anthracis capsule.²² Rates of cleavage of γ-glutamyl donor substrates and transfer of the γ-glutamyl group to an acceptor substrate are critically dependent on the nature of the acceptor: some amino acid acceptor substrates enhanced and some suppressed capsule cleavage, compromising activity in biological milieu and resulting in large differences in enzyme kinetics between simple buffers and complex growth media.²²

The stability of CapD was determined by performing an accelerated storage stability study. Aliquots of enzyme were stored at 40° C. for set time periods, then combined with PDGA, and loss of enzyme activity was measured by viscometry. Because PDGA has an extremely high molecular weight it is very viscous in solution. CapD hydrolyses PDGA, reducing its molecular weight and thus its viscosity. This can be measured by a viscometer, comprising a glass capillary containing a metal ball filled with fluid.

The assay measures the time taken for ball to run from one end of the capillary to the other. If the enzyme has lost activity, PDGA will not be hydrolysed, viscosity will not decrease and run times will be longer.

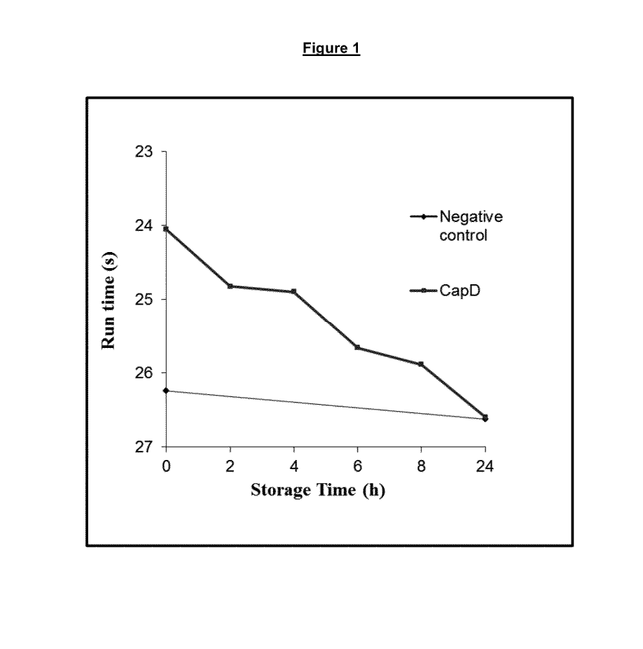

FIG. 1 shows the amount of time CapD was stored at 40 deg C before combining with PDGA and the run time as measured in the viscometer. The results show that by 24 h CapD has lost all activity. The rapid loss of enzyme activity limits viability of CapD as a potential anthrax therapeutic.

We conclude that CapD is not an ideal candidate for development as a therapeutic agent and that more robust deploymerases should be identified and evaluated.

### Example 2

### PDGA Depolymerases from Phages

The presence of a capsule impedes bacteriophage access to the bacterial surface and for this reason the large majority of phages that infect capsule-bearing hosts carry capsule depolymerases that facilitate interaction with the cell surface that underlies the capsular layer.¹⁸

To identify putative phage-encoded PDGA depolymerases, we examined the lytic properties of a range of anthrax phages, including γ, IM, Fah and six recently isolated bacteriophages from the Polish Military Research Centre at Pulawy against a panel of encapsulated and capsule-free B. anthracis phenotypes.¹⁹

More specifically, seven B. anthracis phage (Gamma, IM, Fah, F3, F7, F9, F12) were examined for capacity to degrade high molecular weight PDGA. Of particular interest was gamma phage, which has been reported to infect encapsulated phenotypes of B. anthracis and to do so it would probably possess a capsule depolymerase.

In order to determine degradation of PDGA by B. anthracis phages, aliquots of phage preparation (1×10⁹ pfu) were incubated with PDGA (0-16 h) at 37° C. and polymer degradation products analysed by SDS-PAGE. SDS-PAGE can be used to analyse PDGA degradation because of high molecular weight of PDGA. Undegraded PDGA does not run very far in the gel and stains as a dark band near top. If degraded that band shifts or disappears entirely depending on the degradation products. However, none of the phage preparations were able to degrade PDGA (data not shown), raising the question of whether gamma phage can actually infect encapsulated B. anthracis. Subsequent exoerunebts suggested gamma phage cannot actually infect encapsulated phenotypes of B. anthracis.

### Example 3

### PDGA Depolymerases from Soil Bacteria

To facilitate survival under carbon limiting conditions, many soil bacteria degrade and metabolise a range of diverse carbon-containing compounds as sources of energy. The initial step in the degradation of carbonaceous compounds is most frequently the production of bacterial hydrolytic enzymes which degrade recalcitrant molecules into metabolisable fragments; soil enrichment culture techniques have been used to identify enzymes that degrade pneumococcal capsular polysaccharides,²⁰ indicating the potential of this approach.

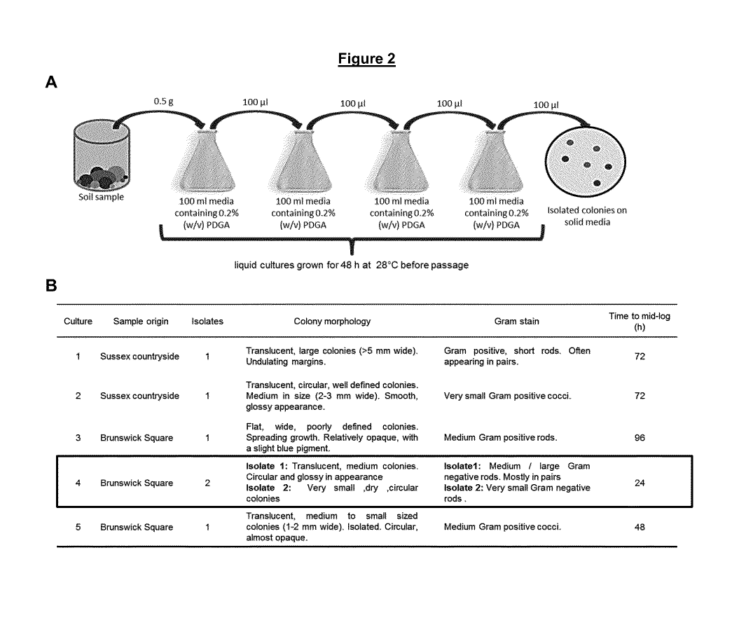

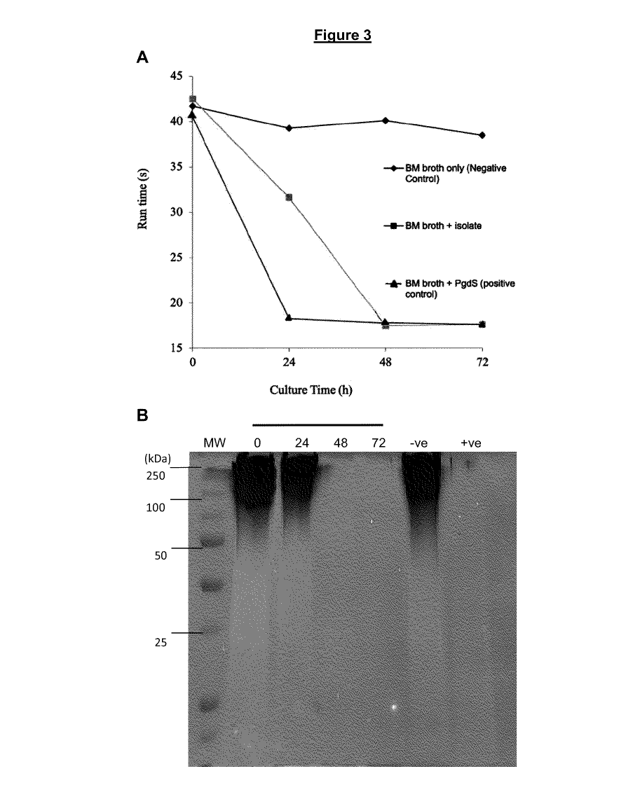

We sampled a variety of soils in Southern England using minimal salts medium containing 0.2% PDGA and isolated a panel of microorganisms able to utilize the polymer as sole source of carbon. Standard soil enrichment techniques were employed to isolate such bacteria, as shown in FIG. 2A.

A number of distinct environmental isolates were found to metabolize PDGA, shown in FIG. 2B. Isolate 4 was chosen as it was fastest growing providing enough material for downstream purifications and experiments.

### Example 4

### Analysis of Isolate 4

Bacteria isolated from culture four identified by sequencing of the 16S rRNA gene.

Isolate 1: Sequence of 16S rRNA gene showed 99% homology with Pseudomonas extremaustralis, Pseudomonas fluorescens, Pseudomonas veronii, & Pseudomonas marginalis

Isolate 2: Identified as a member of the Alcaligenaceae family showing 99% homology with the soil bacterium Pusillimonas noertemannii. Pusillimonas is a recent genus, with only one species having had its genome sequenced.

To check that the mixed culture was degrading PDGA, aliquots of the culture media were taken at various time points and analysed by viscometry and SDS PAGE. As shown in FIG. 3A, by 48 hours, viscosity of culture is same as positive control.

This was confirmed by SDS-PAGE (FIG. 3B) followed by staining with methylene blue, which showed no high-molecular-weight PDGA in the culture medium after 48 h incubation time

### Example 5

### Location of the Cellular Location of the PDGA Depolymerase

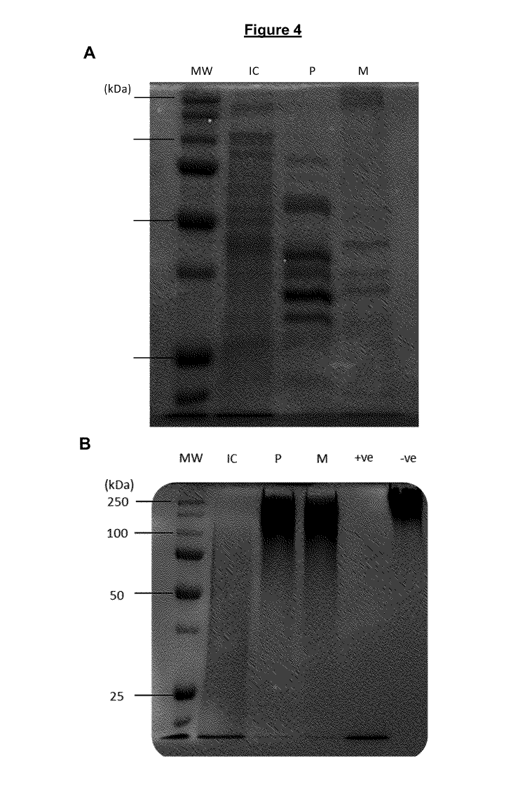

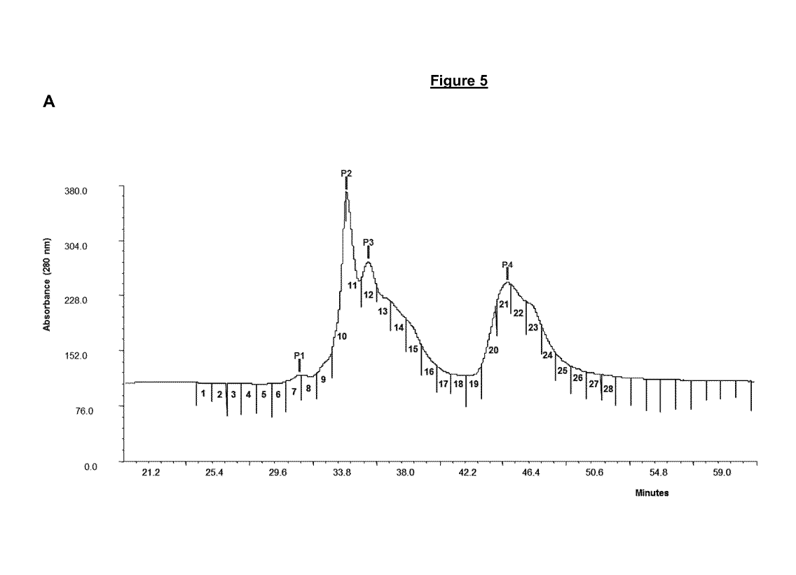

An attempt was made to determine the primary cellular location of the PDGA depolymerase. Cells from the mixed culture were fractioned into intracellular (IC), periplasmic proteins (P) and membrane proteins (M), and the capacity of the cellular fractions to degrade PDGA was determined. Periplasmic proteins were extracted by osmotic shock. Intracellular proteins were released by subsequent sonication of cells followed by isolation of membrane proteins by ultracentrifugation. FIG. 4A shows an SDS-PAGE of the separated fractions, with all lanes loaded with 5 μg of protein.

The capacity of the cellular fractions to degrade PDGA was determined by incubating protein (0.5 μg) from the cellular fractions with PDGA (8 μg) at 37° C. for 16 h. Degradation products were visualized by SDS-PAGE, as shown in FIG. 4B. The results showed a majority of activity was confined to cytoplasmic fraction, however when slightly higher quantity of protein loaded, membrane fraction also had activity.

### Example 6

### Investigation into the PDGA Depolymerase Activity of the Mixed Culture

The characteristics of the mixed culture were examined in more detail, to determine which of the isolates contained a PDGA depolymerase. However, it was found that individual isolates do not grow in minimal medium supplemented with 0.2% (w/v) PDGA as sole source of carbon and energy.

Experiments using 0.2% (w/v) D-glutamic acid as a sole carbon revealed that Pseudomonas can utilise the PDGA monomer for growth however the Pusillimonas cannot. Further, Pseudomonas cultures grown in 0.2% (w/v) D-glutamic acid were found to be devoid of depolymerase activity. These data suggested the depolymerase is not from pseudomonas, as if it was, then the pseudomonas should be able to grow in isolation.

In addition, it was found that growth of the mixed culture in nutrient rich media results in an absence of depolymerase production, indicating that the depolymerase enzyme is inducible.

Together, these data suggest that the PDGA depolymerase enzyme is produced by Pusillimonas, but that Pseudomonas is required for growth of Pusillimonas on PDGA as a sole carbon source.

### Example 7

### Attempt to Purify the Depolymerase

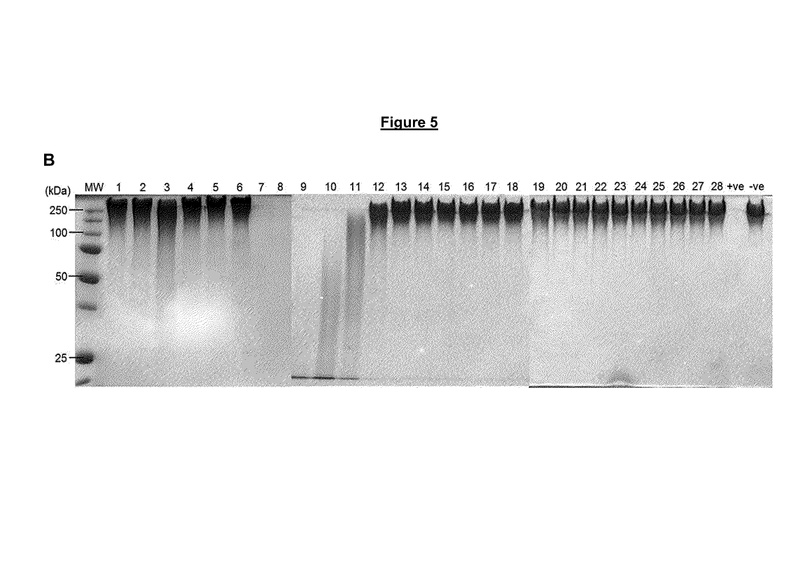

Purification of the depolymerase was attempted using anion exchange chromatography of the intracellular fraction on a HiTrap Q column. Proteins were eluted with a linear gradient of 0-1 M NaCl and fractions (1 ml) were collected in eppendorfs. Four distinct protein peaks were visualised, as shown in FIG. 5A. Individual fractions were concentrated and tested for depolymerase activity. Specifically, protein (0.5 μg) from concentrated individual fractions separated by anion exchange chromatography were incubated with PDGA (8 μg) at 37° C. for 16 h in a 20 μl reaction. Degradation products were visualized by SDS-PAGE stained with methylene blue. As shown in FIG. 5B, fractions 7-9 showed potent activity, and were related to P1 on FIG. 5A.

### Example 8

### Analysis of Semi-Purified Fractions

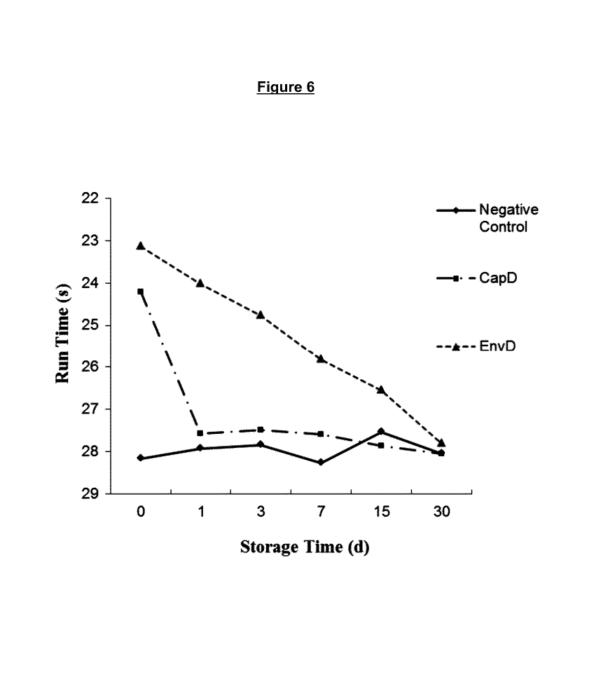

The semi-purified fractions from Example 7 were preliminarily characterised to determine the characteristics of the PDGA depolymerase (termed the environmental depolymerase EnvD). The stability of EnvD was determined as described in Example 1, and compared with CapD. As shown in FIG. 6, EnvD is structurally robust, and the partially purified active fraction retained activity for 15 days upon storage at 40° C.

It was found that EnvD is not a general protease, as no protease activity was found against fibrin or BSA. Further, treatment with commercially available protease inhibitors did not affect hydrolytic activity.

In addition, EnvD was found to have the capacity to catalyse depolymerisation of both L- and D-poly-gamma glutamic acid, and was extremely potent. Semi-purified fractions containing 0.5 μg total protein rapidly (after 1 h) degrade PDGA (8 μg).

### Example 9

### Analysis by Zymography

An attempt was made to identify the actual protein responsible for the depolymerase activity using zymography. Zymography is an electrophoretic technique, based on SDS-PAGE, that includes a substrate (PDGA) copolymerised with the polyacrylamide gel, for the detection of enzyme activity.

Samples are prepared in standard SDS-PAGE buffer but without boiling, and without a reducing agent. Duplicate samples are run on the same gel and following electrophoretic separation, the gel is bisected. This allows one gel half to be developed as a zymogram, elucidating the precise location of the active protein, due to digestion of the incorporated substrate. The duplicate half is stained to visualise all proteins within the sample. The zymogram can then be used as a guide to identify the correlating protein band. The band can then be excised and proteins identified by LC/MS-MS by matching peptides to a protein database. However, this technique requires that the protein is present in a protein database or shares high homology with another protein in the database.

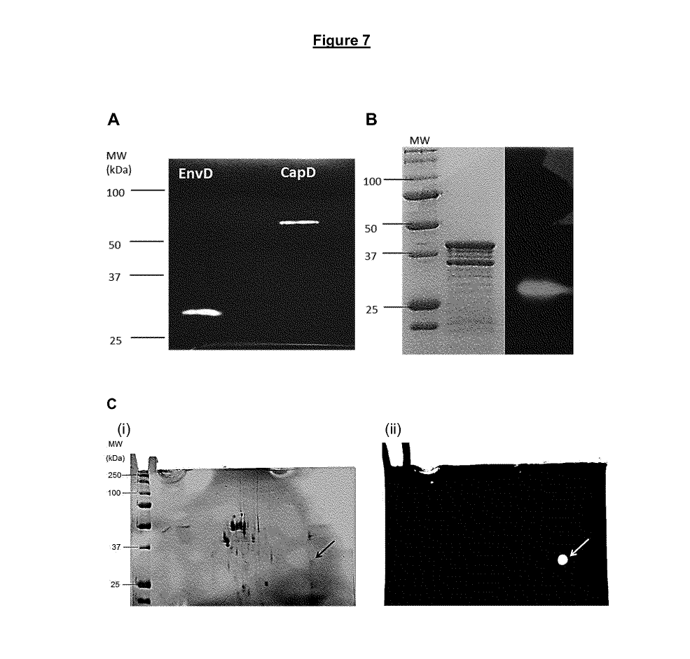

The pooled active fractions from column chromatography were analysed by zymography with CapD as a positive control, as shown in FIG. 7A.

FIG. 7B shows the zymogram aligned next to stained proteins from active fractions run on the same gel under the same conditions. Depolymerase activity is therefore due to a single protein with an approximate molecular weight of 30 kDa. However, there are several bands close to the zone of clearing on the zymogram, and therefore 2D zymography was attempted to achieve better separation of the candidate proteins.

2-D electrophoresis is a technique in which proteins are separated in two dimensions.

Firstly, proteins are separated according to their isoelectric point (pl) by isoelectric focusing (first dimension). Secondly, proteins are separated by molecular weight using SDS-PAGE.

High resolution is achieved due to separation in two dimensions resulting in protein spots with low background contamination. If combined with zymography, by co-polymerizing PDGA with the polyacrylamide gel used in the second dimension, protein spots exhibiting depolymerase activity can be visualised, excised, and identified.

FIG. 7C shows an area of activity identified on the zymogram (ii, indicated by a white arrow), is confined to a small area on the right-hand side of the gel, separated from the majority of proteins. The corresponding area on the Coomassie-stained gel (i) is indicated by the black arrow, and this spot was excised and analysed using LC/MS-MS. The proteins present in the excised spot were identified by LC/MS-MS. Two proteins were identified, as set out below:

| | | | Peptides | |
|---|---|---|---|---|
| | | | matched/ | |
| | | Mass | coverage | |
| Protein | Origin | (kDa) | (%) | Function |
| PFLU1812 | Pseudomonas | 28.5 | 2/10 | putative glycine |
| | fluorescens | | | betaine-binding |
| | SBW25 | | | protein—part of the |
| | | | | ABC transport system |
| PFLU5729 | Pseudomonas | 33 | 4/17 | putative ABC transporter |
| | fluorescens | | | substrate-binding |
| | SBW25 | | | exported protein |

However, neither protein is an enzyme. Further, the proteins were cloned, expressed and tested for depolymerase activity, both were negative.

As noted above, lack of genomic sequence data means identification of proteins from environmental strains is particularly technically difficult, although as explained below even with genomic data the identification was not straightforward.

### Example 10

### Genomic Analysis

Genomes of both organisms were sequenced in collaboration with LSHTM (Richard Stabler) and KAUST (Arnab Pain), as described in Reference 21. The genomic sequences referred to therein as AMZF00000000 and AMZG00000000 are hereby specifically incorporated by reference.

Whole-genome sequencing was performed on the Illumina Hiseq2000 platform using a single read (read 1) from a paired-end read library of read length 100 bp.

Pseudomonas fluorescens BS2 genome was found to be 6.1 Mbp and P. noertemannii BS8 was found to be 3.9 Mbp

Automated gene prediction and annotation were performed using RAST (Rapid Annotation using Subsystem Technology), which predicted 5539 and 3633 coding sequences for P. fluorescens BS2 and P. noertemannii BS8, respectively. This was used to construct a new protein database.

Interestingly, RAST indicated that the nearest neighbour to P. fluorescens BS2 was P. fluorescens SBW25 and to P. noertemannii BS8 was Bordetella bronchiseptica RB50.

### Example 11

### Interrogation of Protein Database

Spectra generated from the previous 2D LC-MS/MS analysis of Example 9 were used to probe newly constructed protein databases (unpublished).

Over 50 proteins identified for P. fluorescens BS2 by peptide matching, and putative functions for each protein identified by RAST and BLAST. High genome homology with P. fluorescens SBW25 meant a number of proteins had been identified previously and cloned.

Over 50 proteins identified for P. noertemannii BS8 by peptide matching, and putative functions for each protein identified by RAST and BLAST.

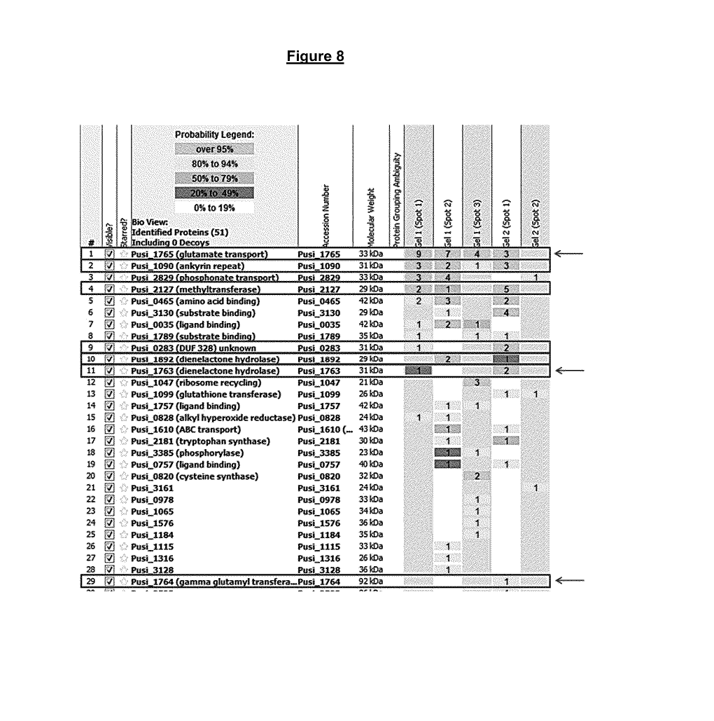

Proteins were selected for characterisation based on criteria including: predicted function, correct molecular weight and relative quantity, as shown in FIG. 8.

### Example 12

### Identification of Gene Encoding EnvD

The cloned Pusillimonas genes were tested for depolymerase activity by SDS-PAGE, as described above.

Proteins 0208 (DUF 328), 1090 (ankyrin repeat), 1892 (dienelactone hydrolase) and 2127 (methyltransferase) were cloned, expressed and examined for activity. All were negative.

Proteins 1763 (dienelactone hydrolase), 1764 (gamma-glutamyl-transferase) and 1765 (glutamate transport protein) were cloned and tested together as they appeared to form part of an operon involved in glutamic acid metabolism.

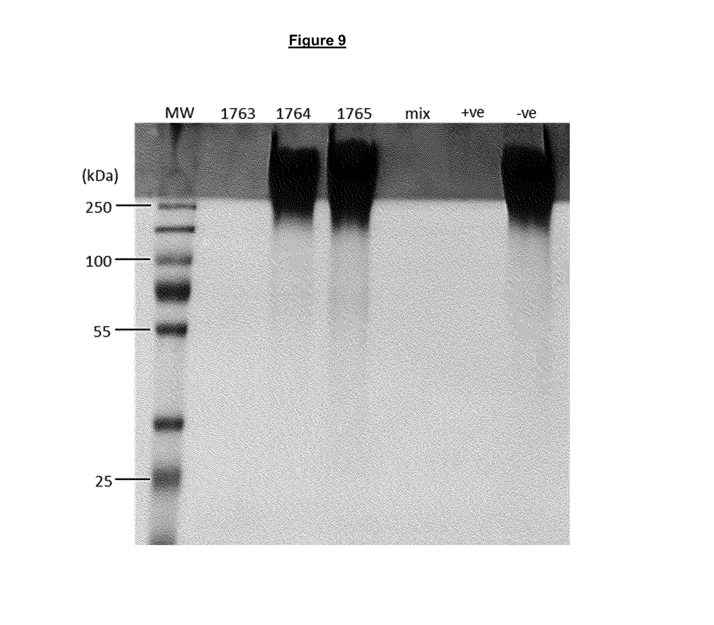

Protein 1763 (putative dienelactone hydrolase) was found to possess potent depolymerase activity (shown in FIG. 9) but is highly insoluble. The mix of proteins 1763, 1764 and 1765 similarly showed potent depolymerase activity.

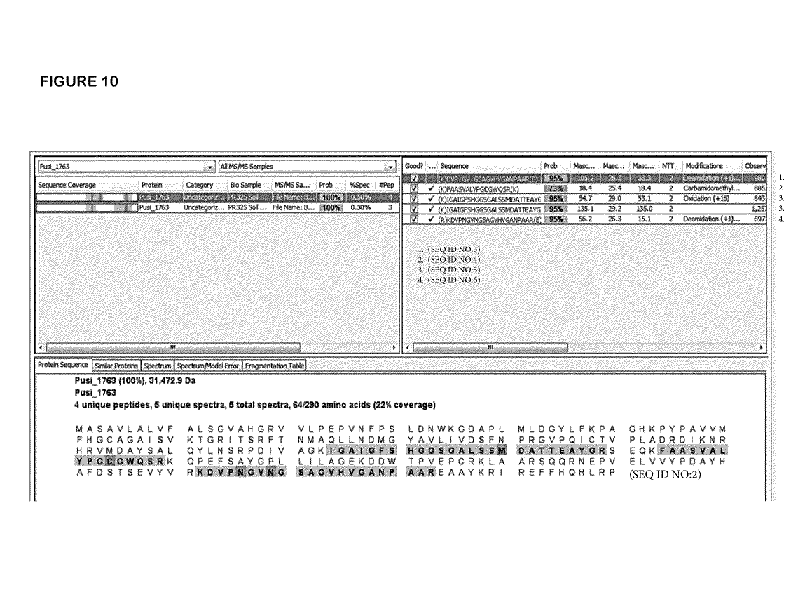

EnvD was therefore identified as protein 1763. FIG. 10 shows an image of the exact peptides that were matched to the protein using LC/MS-MS. The nucleotide sequence of EnvD is described herein as SEQ ID NO:1, and the amino acid sequence is described herein as SEQ ID NO: 2.

### Example 13

### Analysis of EnvD Primary Structure

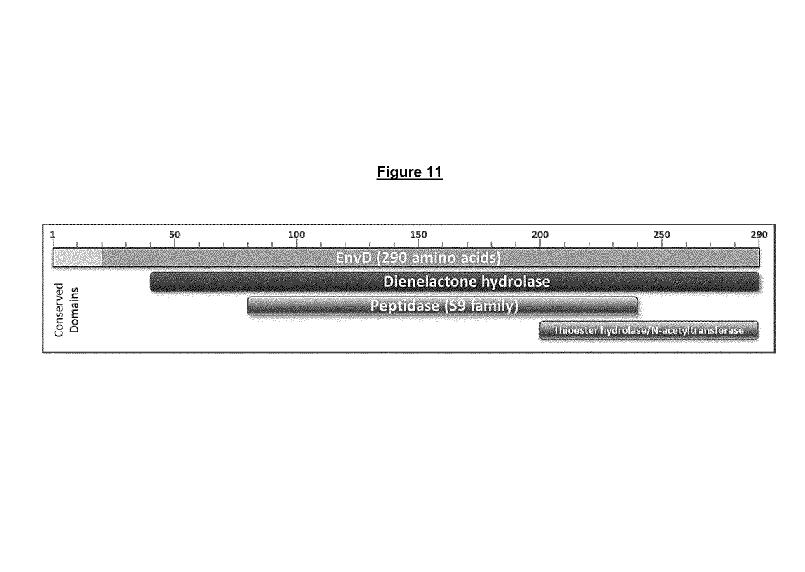

To find out more about EnvD, including how it may hydrolyze PDGA, several free online protein analysis tools were used to analyse the amino acid sequence. FIG. 11 shows conserved domains identified on EnvD. Multi-domain models are shown in dark blue (i.e. dienelactone hydrolase) and contain hits for multiple single domains. Non-specific hits are shown in red (i.e. peptidase [S9 family] and Thioester hydrolase/N-acetyltransferase) and represent domains that meet or exceed the E-value cut-off for statistical significance (0.01). An N-terminal signal peptide comprising of 18 amino acids is identified in light blue.

The primary sequence of EnvD indicates that it contains motifs consistent with peptidase activity, a more desirable mode of action in comparison to CapD.

Peptidase family S9 contains a varied set of serine-dependent peptidases, including acylaminoacyl-peptidase (EC 3.4.19.1). Acylaminoacyl-peptidase is an omega-peptidase that releases N-acylated amino acids from oligopeptides. Interestingly, omega-peptidases have the capacity to hydrolyze peptide bonds that are not alpha-bonds, including gamma-bonds.^{30,31}.

The conserved S9 active site residues Ser, Asp and His are at positions 150, 208 and 240 respectively.

### Example 14

### Model for Inhalation Anthrax

An exemplary assay can be performed as follows:

Groups of ten BALB/C mice (female, adult over ten weeks old) are challenged with a target dose of 10-50× minimum lethal dose equivalents (MLDs) of B. anthracis Ames strain using the AeroMP-Henderson apparatus. The challenge aerosol is generated using a Collison nebuliser, mixed with conditioned air in the spray tube³⁷ and delivered to the nose of each animal via the exposure tube. Samples of the aerosol are obtained using an AGI30 glass impinger and mean particle size determined with a TSI Aerodynamic Particle Sizer; these processes are controlled and monitored using the AeroMP management platform. Two concentrations of the EnvD-based composition are administered by the intra-peritoneal and intra-nasal route 1 h before challenge and 1 h, 4 h and 12 h post-challenge and then daily for a further two days should the animals survive. Control groups include a challenge only, a sham-treated/challenged and a treated/sham-challenged cohort for each administration route. Animals are monitored and assigned a clinical score at least twice daily over the fourteen day post-challenge period. Post mortem, samples of blood, lung and spleen are taken for enumeration of bacterial load and samples of lung and spleen are placed in 10% neutral-buffered formalin for evaluation of pathological changes. Fixed tissues are processed to paraffin wax, sections cut at 4 μm and stained with haematoxylin and eosin.

Treatment efficacy is assessed in terms of survival using Kaplan-Meier with Log-Rank statistics; reduction of bacterial load and pathological changes may provide further endpoints for evaluation of the efficacy of the composition.

### REFERENCES

[1] Mock M & Fouet A (2001) Ann. Rev. Microbiol. 55:647-671;
[2] Franz D R, Jahrling P B, Friedlander A M, McClain D J, Hoover D L, Bryne W R, Pavlin J A, Christopher G W & Eitzen E M (1997) J. Amer. Med. Assoc. 278:399-411;
[3] Inglesby T V, Henderson D A, Bartlett J G, Ascher M S, Eitzen E, Friendlander A M, Hauer J, McDade J, Osterholm M T, O'Toole T, Parker G, Perl T M, Russell P K & Tonat K (1999) J. Amer. Med. Assoc. 281:1735-1745;
[7] Koehler T M (2002) Curr. Top. Microbiol. Immunol. 271:143-164;
[8] Drysdale M, Heninger S, Hutt J, Chen Y, Lyons C R & Koehler T M (2005) EMBO J. 24:221-227;
[9] Dixon T C, Mesleson M, Guillemin J & Hanna P C (1999) New Engl. J. Med. 341:815-826;
[10] Mushtaq N, Redpath M B, Luzio J P & Taylor P W (2004) Antimicrob. Agents Chemother. 48:1503-1508;
[11] Mushtaq N, Redpath M B, Luzio J P & Taylor P W (2005) J. Antimicrob. Chemother. 56:160-165;
[12] Zelmer A, Bowen M, Jokilammi A, Finne J, Luzio J P & Taylor P W (2008) Microbiology 154:2522-2532;
[13] Zelmer A, Martin M, Gundogdu O, Birchenough G, Lever R, Wren B W, Luzio J P & Taylor P W (2010) Microbiology 156:2205-2215;
[14] Candela T & Fouet A (2005) Molec. Microbiol. 57:717-726;
[15] Scorpio A, Chabot D J, Day W A, O'Brien D K, Vietri N J, Itoh Y, Mohamadzadeh M & Friedlander A M (2006) Antimicrob. Agents Chemother. 51:215-222;
[16] Scorpio A, Tobery S A, Ribot W J & Friedlander A M (2008) Antimicrob. Agents Chemother. 52:1014-1020;
[17] Wu S J, Eiben C B, Carra J H, Huang I, Zong D, Liu P, Wu C T, Nivala J, Dunbar J, Huber T, Senft J, Schokman R, Smith M D, Mills J H, Friedlander A M, Baker D & Siegel J B (2011) J. Biol. Chem. 286:32586-32592;
[18] Sutherland I W, Hughes K A, Skillman L C & Tait K (2004) FEMS Microbiol. Lett. 232:1-6;
[19] Negus D, Burton J, Sweed A, Gryko R & Taylor P W (2013) Appl. Environ. Microbiol. doi:10.1128/AEM.02682-12;
[20] Dubos R & Avery O T (1931) J. Exp. Med. 54:51-71;
[21] Stabler R A, Negus D, Pain A & Taylor P W (2013) Genome Announc. 1:e00057-12;
[22] Hu X, Legler P M, Khavrutski I, Scorpio A, Compton J R, Robertson K L, Friedlander A M & Wallqvist A (2012) Biochemistry 51:1199-1212;
[23] Lilie H, Schwarz E & Rudolph R (1998) Curr. Opin. Biotechnol. 9:497-501;
[24] Tolia N H & Joshua-Tor L (2006) Nat. Meth. 3:55-64;
[25] Sun P, Tropea J E & Waugh D S (2011) Meth. Molec. Biol. 705:259-274;
[26] Ferre F & Clote P (2005) NucL Acids Res. 33:W230-2;
[27] Petersen T N, Brunak S, von Heijne G & Nielsen H (2011) Nat. Meth. 8:785-786;
[28] Bessette P H, Aslund F, Beckwith J & Georgiou G (1999) Proc. Natl. Acad. Sci. U.S.A. 96:13703-13708;
[29] Leggate D R, Bryant J M, Redpath M B, Head D, Taylor P W & Luzio J P (2002) Molec. Microbiol. 44:749-760;
[30] McDonald J K (1985) Histochem. J. 17:773-785;
[31] Polgár L (2002) Cell Mol. Life Sci. 59:349-362;
[32] Sutherland M D & Kozel T R (2009) Infect. Immun. 77:532-538;
[33] Suzuki T & Tahara Y (2003) J. Bacteriol. 185:2379-2382;
[34] Rawlings N D, Tolle D P & Barrett A J. (2004) Biochem. J. 378:705-716;
[35] Fülöp V, Böcskei Z & Polgár L (1998) Cell 94:161-170;
[36] Klock H E & Lesley S A (2009) Meth. Mol. Biol. 498:91-103;
[37] Druett H A (1969) J. Hyg. (Lond.) 67:437-48

1. A PGDA depolymerase polypeptide having a sequence which is at least about 60%, 70%, 80% or 85% identical to the amino acid sequence provided herein as SEQ ID NO: 2, which comprises a His tag or which is a fusion protein of SEQ ID NO: 2, or a fragment thereof with a heterologous fusion partner.

2. (canceled)

3. A PGDA depolymerase polypeptide comprising, consisting of, or consisting essentially of, the amino acid sequence provided herein as SEQ ID NO: 2, or a fragment thereof, which lacks all or part of the signal peptide shown as amino acids 1 to 18 of SEQ ID NO: 2.

4.-9. (canceled)

10. A vector comprising a nucleic acid having a sequence selected from the group consisting of:
(a) a sequence encoding a PGDA depolymerase polypeptide having a sequence which is at least about 60%, 70%, 80% or 85% identical to the amino acid sequence provided herein as SEQ ID NO: 2;
(b) a sequence which is complementary to, or which hybridises to, a sequence as defined in (a);
(c) a fragment of a sequence as defined in (a) or (b); and
(d) a sequence that is degenerate as a result of the genetic code to any one of (a) to (c); wherein the sequence is operably linked to a heterologous promoter.

11. A host cell transformed with the vector of claim 10.

12. A host cell as claimed in claim 11 wherein said cell is prokaryotic, and which is preferably E. coli.

13. A host cell as claimed in claim 12 which possesses mutations in thioredoxin reductase and\or glutathione reductase genes.

14. A method of producing a host cell having enhanced PGDA depolymerase activity, which method comprises introducing into said host a vector as claimed in claim 10.

15. A process of producing a polypeptide, the process comprising culturing a host cell as claimed in claim 11 under conditions that provide for expression of the polypeptide encoded by the sequence.

16. A process as claimed in claim 15 which comprises the steps of:
(i) culturing said host cell in a suitable culture medium,
(ii) causing expression of said vector sequence as defined above under suitable conditions for production of soluble protein and optionally,
(iii) lysing said transformed host cells and recovering said polypeptide, or recovering secreted polypeptide from the culture medium.

17. A composition comprising a PGDA depolymerase polypeptide having a sequence which is at least about 60%, 70%, 80% or 85% identical to the amino acid sequence provided herein as SEQ ID NO: 2, which is in freeze-dried form.

18.-19. (canceled)

20. A pharmaceutical or veterinary composition comprising a PGDA depolymerase polypeptide having a sequence which is at least about 60%, 70%, 80% or 85% identical to the amino acid sequence provided herein as SEQ ID NO: 2, and a pharmaceutically or veterinarily acceptable carrier.

21. A composition as claimed in claim 20 which is suitable for parenteral administration.

22. A composition as claimed in claim 20 which is in liquid form.

23. A method of treating an infection in a subject, said infection produced by an organism having a capsule comprising a PGDA polymer, said method comprising introducing into said subject a PGDA depolymerase polypeptide having a sequence which is at least about 60%, 70%, 80% or 85% identical to the amino acid sequence provided herein as SEQ ID NO:2, in a therapeutically effective amount.

24. A method as claimed in claim 23 wherein the treatment is prophylactic treatment.

25. A method as claimed in claim 23 wherein said organism is B. anthracis.

26. A method as claimed in claim 23 wherein the infection is inhalation anthrax.

27. A method as claimed in claim 23 further comprising administering to said subject an antibiotic.

28. (canceled)

29. A method as claimed in claim 27 wherein said antibiotic is ciprofloxacin.

30.-32. (canceled)

33. An antibody molecule specific for a polypeptide of claim 3.
